(19) United States
(12) Patent Application Publication
(10) Pub. No.: US 2026/0216033 A1
(43) Pub. Date: Jul. 30, 2026

(54) Eyebrow Make-Up Composition with a Non-Glycerolated Silicone Resin, a Glycerolated Silicone Resin, a Volatile Hydrocarbon Oil and a Thickener

(71) Applicant: L'OREAL, PARIS (FR)

(72) Inventors: ALEXIS LIARD, SAINT-OUEN (FR); MARINE MICHEL, CHEVILLY-LARUE (FR)

(73) Assignee: L'OREAL, Paris (FR)

(21) Appl. No.: 19/146,608

(22) PCT Filed: Dec. 13, 2023
(86) PCT No.: PCT/EP2023/085667

§ 371 Date: Jul. 9, 2025

(30) Foreign Application Priority Data
Jan. 10, 2023 (FR) FR2300240

(51) Int. Cl. A61K 8/58; A61K 8/92; A61Q 1/10

(52) U.S. Cl. CPC A61K 8/585; A61K 8/92; A61Q 1/10; A61K 2800/43; A61K 2800/622; A61K 2800/88

(57) ABSTRACT

The present invention relates to an anhydrous composition for caring for and/or making up keratin materials, in particular the eyebrows and the skin around the eye and eyebrows, comprising, in particular in a physiologically acceptable medium: a) at least one non-glycerolated silicone resin; and b) at least one glycerolated silicone resin; and c) at least one oily phase comprising at least one volatile hydrocarbon; and d) a lipophilic thickener. It also relates to a method for coating keratin materials, in particular the eyebrows and the skin around the eye and eyebrows, and more particularly to a method for making up said keratin materials, comprising the application thereto of the composition as defined above.

The present application relates to the field of making up keratin materials, in particular eyebrows including the eyebrow hairs, the skin for implantation of said hairs and the contours thereof.

In the field of eyebrow make-up, consumers have access to several types of solutions:
eyebrow pencils, which are easy to use but only last for a day. They are often based on a pigmented lead which colours by transferring material onto the skin.
pens, which are also easy to use but only last for a day. They are often composed of aqueous formulations containing dyes.
in-salon tattooing services, which are very painful, but last for several months.
anhydrous gels such as the commercial products Inked Waterproof Brow Gel® from Urban Decay (Mintel ID 7578707) and Up to 3 Day Styling Gel® from Maybelline (Mintel ID 10361806) comprising isododecane and the combination of an MQ resin: TRIMETHYLSILOXYSILICATE and a silicone polyamide: NYLON-611/DIMETHICONE COPOLYMER.

Users of make-up formulations for keratin materials such as the eyebrows and the skin around the eye and eyebrows are looking for products with a longer wear property over time, which is reflected in particular by a better resistance of the deposited film to sebum, which is a complex mixture of lipids synthesized under hormonal stimulation by the sebaceous glands present in the dermis. When sebum is present on the skin around the eye and eyebrows, it tends to lessen the wear property of the eyebrow makeup.

The need remains to find novel compositions for caring for and/or making up keratin materials, in particular eyebrows and the skin around the eye and the eyebrows, which make it possible to obtain a makeup having a better wear property of the deposit over time, in particular a better resistance to sebum.

Unexpectedly, the inventors have found that it is possible to achieve these objectives by using a composition, preferably for caring for and/or making up keratin materials, in particular eyebrows including the eyebrow hairs, the skin for implantation of said hairs and the contours thereof, comprising, in particular in a physiologically acceptable medium:
a) at least one non-glycerolated silicone resin of MQ type; and
b) at least one glycerolated silicone resin; and
c) at least one oily phase comprising at least one volatile hydrocarbon oil; and
d) a lipophilic thickener.

This discovery forms the basis of the invention.

### SUBJECTS OF THE INVENTION

Thus, a first subject of the present invention is a composition, preferably for caring for and/or making up keratin materials, in particular eyebrows including the eyebrow hairs, the skin for implantation of said hairs and the contours thereof, comprising, in particular in a physiologically acceptable medium:
a) at least one non-glycerolated silicone resin of MQ type; and
b) at least one glycerolated silicone resin; and
c) at least one oily phase comprising at least one volatile hydrocarbon oil; and
d) a lipophilic thickener.

A second subject of the present invention is a method for coating keratin materials, in particular the eyebrows and the skin around the eye and eyebrows, and more particularly a method for making up said keratin materials, comprising the application thereto of the composition as defined above.

### Definitions

In the context of the present invention, the term “keratin material” is notably intended to mean the eyebrows including the eyebrow hairs, the skin for implantation of said hairs and the contours thereof. For the purposes of the present invention, this term “keratin materials” also extends to synthetic false eyebrows.

The term “physiologically acceptable” means compatible with said keratin materials, which has a pleasant colour, odour and feel, and which does not cause any unacceptable discomfort (stinging or tautness) liable to discourage the consumer from using this composition.

The term “glycerolated silicone resin” is understood to mean any silicone resin comprising at least one organosiloxane unit comprising one or more monoglycerol or polyglycerol groups in its chemical structure.

In particular, the glycerolated silicone resin contains at least one organosiloxane unit of the RR′R″SiO_{1/2} type in which R, R′ and R′″, which are identical or different, denote hydrocarbon radicals, of which at least one of said radicals contains a monoglycerol group or a polyglycerol group, and more particularly the glycerolated silicone resin contains at least one dimethylsiloxane R(CH₃)₂SiO_{1/2} unit comprising a hydrocarbon radical R comprising a monoglycerol group.

The term “hydrocarbon radical” is understood to mean a radical containing predominantly hydrogen and carbon atoms and optionally one or more functions chosen from hydroxyl, ester, ether and carboxyl functions.

The term “monoglycerol group” is understood to mean any group comprising in its chemical structure a —O—CH₂—CHOH—CH₂OH group.

The term “polyglycerol group” is understood to mean any group comprising in its chemical structure a chain comprising a repetition of at least 2 —(O—CH₂—CHOH—CH₂)ₘ glycerol units.

### Non-Glycerolated Silicone Resin

The composition according to the invention comprises at least one non-glycerolated silicone resin of MQ type.

More generally, the term resin means a compound whose structure is three-dimensional. Silicone resins are also known as “siloxane resins”. Thus, for the purposes of the present invention, a polydimethylsiloxane is not a silicone resin.

The nomenclature of silicone resins (also known as siloxane resins) is known under the name “MDTQ”, the resin being described as a function of the various siloxane monomer units it comprises, each of the letters MDTQ characterizing a type of unit.

The letter M represents the monofunctional unit of formula R₁R₂R₃SiO_{1/2}, the silicon atom being connected to only one oxygen atom in the polymer comprising this unit.

The letter D means a difunctional unit R₁R₂SiO_{2/2} in which the silicon atom is connected to two oxygen atoms.

The letter T represents a trifunctional unit R₁SiO_{3/2}.

Such resins are described, for example, in the Encyclopedia of Polymer Science and Engineering, vol. 15, John Wiley & Sons, New York, (1989), pp. 265-270, and U.S. Pat. Nos. 2,676,182, 3,627,851, 3,772,247, 5,248,739 or U.S. Pat. Nos. 5,082,706, 5,319,040, 5,302,685 and 4,935,484.

In the units M, D and T defined previously, R, namely R₁, R₂ and R₃, represents a hydrocarbon-based radical (notably alkyl radical) containing from 1 to 10 carbon atoms, a phenyl group, a phenylalkyl group or else a hydroxyl group.

Finally, the letter Q means a tetrafunctional unit SiO₄₁₂ in which the silicon atom is bonded to four oxygen atoms, which are themselves bonded to the rest of the polymer.

### MO Resins:

As examples of silicone resins of MQ type, mention may be made of the alkylsiloxysilicates of formula [(R₁)₃SiO_{1/2}]ₓ(SiO_{4/2})_{y} (MQ units) in which x and y are integers ranging from 50 to 80, and such that the group R₁ represents a radical as defined previously, and is preferably an alkyl group containing from 1 to 8 carbon atoms or a hydroxyl group, preferably a methyl group.

As examples of MQ silicone resins of trimethyl siloxysilicate type, mention may be made of those sold under the reference SR1000® by the company General Electric, under the reference TMS 803® by the company Wacker, or under the name KF-7312J® by the company Shin-Etsu or DC749® or DC593® by the company Dow Corning.

The composition according to the invention comprises, as silicone resin, at least one resin of MQ type, more particularly of trimethylsiloxysilicate type, such as those sold under the reference SR1000® by the company General Electric, under the reference TMS 803® by the company Wacker, or under the name KF-7312J® by the company Shin-Etsu or DC749® or DC593® by the company Dow Corning, under the reference SILSOFT 74 FLUID® by the company MOMENTIVE PERFORMANCE MATERIALS.

Use will particularly be made of a trimethylsiloxysilicate resin in solution in isododecane, in particular in a solution containing 75% by weight of active material in isododecane, such as the commercial product sold under the reference SILSOFT 74 FLUID® by the company MOMENTIVE PERFORMANCE MATERIALS.

According to a particular embodiment of the invention, the non-glycerolated silicone resin(s) is (are) present in the composition in an active material content ranging from 4% to 35% by weight relative to the total weight of the composition, preferably ranging from 6% to 30% by weight and more preferentially from 8% to 25% by weight relative to the total weight of the composition.

### Glycerolated Silicone Resin

The composition according to the invention comprises at least one glycerolated silicone resin.

The glycerolated silicone resin comprises in its chemical structure one or more monoglycerol or polyglycerol groups.*

According to a particular embodiment of the invention, the glycerolated silicone resin(s) is (are) present in an active material content ranging from 0.1% to 40% by weight relative to the total weight of the composition, preferably ranging from 0.2% to 30% by weight and more preferentially from 0.5% to 15% by weight relative to the total weight of the composition.

The glycerolated silicone resin(s) according to the invention are preferably chosen from those of formula (I) below:

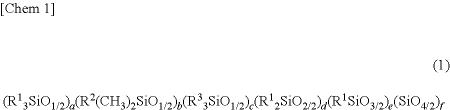

in which
each R¹, which are identical or different, is an alkyl, aryl or aralkyl group with 1 to 30 carbon atoms, or a group substituted by a halogen, a group substituted by an amino or a group substituted by a carboxyl of the latter;
each R² is a monoglycerol or polyglycerol group of general formula (2) below:

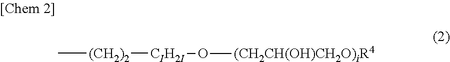

in which
R⁴ is a substituted or unsubstituted monovalent hydrocarbon group or a hydrogen atom, and
the subscripts l and i are integers which satisfy the conditions 0≤l≤15 and 0<i≤5,
each R³ is an identical or different group of general formula (3), of general formula (4), of general formula (5) or of general formula (6) below

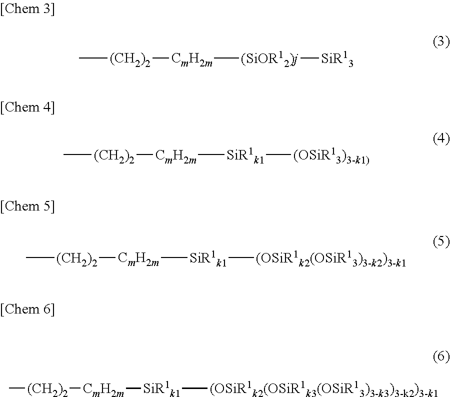

where
each R¹, which are identical or different, is an alkyl, aryl or aralkyl group with 1 to 30 carbon atoms, or a group substituted by a halogen, a group substituted by an amino or a group substituted by a carboxyl of the latter;
the subscripts m, j and k₁ to k₃ are integers which satisfy the conditions 0≤m≤5, 0≤j≤500, 0≤k1≤2, 0≤k₂≤2 and 0≤k₃≤2;
the subscripts a, b, c, d, e and f are numbers which satisfy the conditions 0≤a≤400, 0<b≤200, 0≤c≤400, 0≤d≤320, 0≤e≤320, 0≤f≤1000 and 0.5≤(a+b+c)/f≤1.5.

The glycerolated silicone resins according to the invention are described in patent application US20200332065A1 by SHIN ETSU.

According to a particular embodiment, the glycerolated silicone resin(s) of formula (I) as defined above are chosen from those for which
the subscripts b and c satisfy the conditions 0<b≤30 and 0≤c≤30;
the subscript i in the general formula (2) of the monoglycerol or polyglycerol group R² is an integer that satisfies the condition 0<i≤3.

According to a particular embodiment, the glycerolated silicone resin(s) of formula (I) are in solid form at 25° C. when the subscript c satisfies the condition 0<c≤400 and R³ is a group of general formula (3) where the subscript j satisfies the condition 0≤j≤10.

According to a particular embodiment, the glycerolated silicone resin(s) have a weight-average molecular weight ranging from 1000 to 100 000.

The glycerolated silicone resin(s) according to the invention are amphiphilic, that is to say have two parts of different polarity. Generally, one is lipophilic (soluble or dispersible in an oily phase). The other is hydrophilic (soluble or dispersible in water). They are characterized by the value of their HLB (hydrophilic-lipophilic balance), the HLB being the ratio of the hydrophilic part to the lipophilic part in the molecule. The term “HLB” is well known to those skilled in the art and is described, for example, in “The HLB system. A Time-Saving Guide to Emulsifier Selection” (published by ICI Americas Inc.; 1984). The value of the HLB of the glycerolated silicone resins according to the invention preferably varies from 0.1 to 15 according to the Griffin method.

The glycerolated silicone resin(s) according to the invention may be obtained by a preparation process comprising the step of hydrosilylation:
A) of a silicone resin containing a hydrosilyl group of formula (7) below:

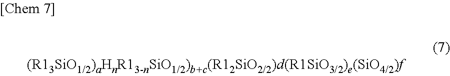

in which:
   each R¹, which are identical or different, is an alkyl, aryl or aralkyl group with 1 to 30 carbon atoms, or a group substituted by a halogen, a group substituted by an amino or a group substituted by a carboxyl of the latter;
   the subscripts a, b, c, d, e and f are integers that satisfy the conditions 0≤a≤400, 0<b≤200, 0≤c≤400, 0≤d≤320, 0≤e≤320, 0<f≤1000 and 0.5≤(a+b+c)/f≤1.5;
   n is an integer which satisfies the condition 1≤n≤3, with
B) one or more compounds which are chosen from the compounds terminated by an alkenyl group of general formulae (8), (9), (10), (11) and (12) below:

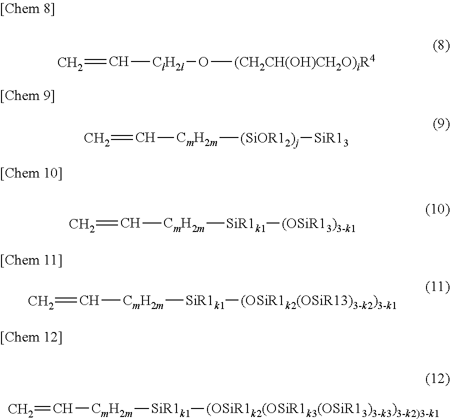

where
   R⁴ is a substituted or unsubstituted monovalent hydrocarbon group or a hydrogen atom,
   the subscripts l and i are integers that satisfy the conditions 0≤l≤15, 0<i≤5;
   the subscripts m, j and k1 to k3 are integers that satisfy the conditions 0≤m≤5, 0≤j≤500, 0≤k1≤2, 0≤k2≤2 and 0≤k3≤2; said silicone resin containing a hydrosilyl group of formula (7) which reacts with at least one compound of formula (8).

The hydrosilylation reaction is carried out in the presence, for example, of a platinum or rhodium catalyst. The preferred ranges for b, c, d, e, f, R4, l, m, i, j and k1 to k3 are as defined above.

### Silicone Resin Containing a Hydrosilyl Group Used as Starting Material.

The silicone resin containing a hydrosilyl group of formula (7) may be in a solid or liquid form at 25° C., although, in terms of film-forming ability, it is preferably solid. From the point of view of utility, the resin is preferably diluted with an organic solvent. The use of a solvent having a boiling point higher than the reflux temperature during the hydrolysis is preferred.

Among the examples of organic solvents used for the dilution, mention may be made of cyclic organopolysiloxanes such as octamethylcyclotetrasiloxane, decamethylcyclopentasiloxane and dodecamethylcyclohexasiloxane; aromatic hydrocarbons such as toluene and xylene; organic solvents of ketone type such as acetone, methyl ethyl ketone, diethyl ketone and methyl isobutyl ketone; aliphatic hydrocarbons such as hexane, heptane, octane and cyclohexane; and aliphatic alcohols such as methanol, ethanol, 1-propanol, 2-propanol, 1-butanol, 2-methyl-1-propanol, 2-butanol, 2-methyl-2-propanol, 1-pentanol, 2-methylbutanol, 2-pentanol, 1-hexanol, 2-methylpentanol, 1-heptanol, 1-octanol, 1-nonanol, 1-decanol, phenol, benzyl alcohol, ethylene glycol and 1,2-propylene glycol. From the point of view of storage stability and absence of volatility, octamethylcyclotetrasiloxane and decamethylcyclopentasiloxane are preferred.

The silicone resin containing a hydrosilyl group of formula (7) is prepared: (i) by hydrolysing, in the presence of an acid catalyst, a mixture of one or more compounds chosen from the organosilicon compounds of general formulae (13) and (14) below, one or more compounds chosen from the organosilicon compounds containing a hydrosilyl group of the general formulae (15) and (16) below and one or more compounds chosen from the hydrolysable silanes of general formula (17) below, the partial hydrolytic condensates of these hydrolysable silanes and the metal salts of these hydrolysable silanes.

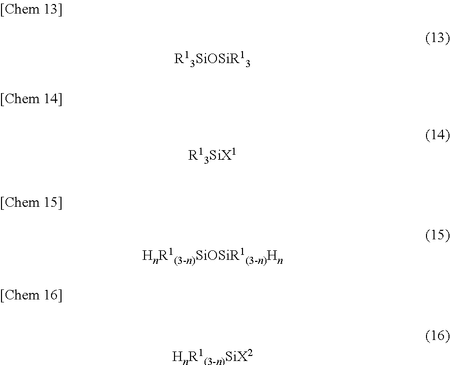

where
each R¹, which are identical or different, is an alkyl, aryl or aralkyl group with 1 to 30 carbon atoms, or a group substituted by a halogen, a group substituted by an amino or a group substituted by a carboxyl of the latter;
X¹ and X² are hydrolysable functional groups; and
n satisfies the condition 1 n 3.

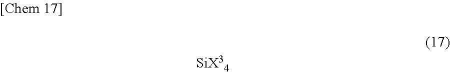

where X³ is a hydrolysable functional group),
ii) by neutralizing the reaction system by adding a basic catalyst in an amount greater than the molar equivalent of the acid catalyst, and
iii) by then carrying out a condensation.

In the general formulae (13), (14), (15) and (16), the examples and the preferred range for R¹ are the same as those mentioned above.

In the general formula (14), X¹ is a hydrolysable functional group which is directly bonded to a silicon atom. Examples include halogen atoms such as chlorine and bromine atoms, alkoxy groups such as methoxy, ethoxy, propoxy and butoxy groups, alkenoxy groups, acyloxy groups, amide groups and oxime groups. Among these, from the point of view of availability and the rate of hydrolysis, a methoxy group, an ethoxy group or a chlorine atom is preferred.

In the general formula (16), X² is a hydrolysable functional group which is directly bonded to a silicon atom. Examples include halogen atoms such as chlorine and bromine atoms, alkoxy groups such as methoxy, ethoxy, propoxy and butoxy groups, alkenoxy groups, acyloxy groups, amide groups and oxime groups. Among these, from the point of view of availability and rate of hydrolysis, a methoxy group, an ethoxy group or a chlorine atom is preferred.

In the general formula (17), X³ is a hydrolysable functional group which is directly bonded to a silicon atom. Examples include halogen atoms such as chlorine and bromine atoms, alkoxy groups such as methoxy, ethoxy, propoxy and butoxy groups, alkenoxy groups, acyloxy groups, amide groups and oxime groups. Among these, an alkoxy group is preferred; from the point of view of availability and the rate of hydrolysis, a methoxy group or an ethoxy group is preferred. The hydrolysable groups X³ on the molecule may be similar or different groups.

Examples of organosilicon compounds of general formula (13) include 1,1,1,3,3,3-hexamethyldisiloxane, 1,1,1,3,3,3-hexaphenyldisiloxane, 1,1,3,3-tetramethyl-1,3-divinyldisiloxane, 1,1,1,3,3,3-hexaethyldisiloxane, 1,1,1,3,3,3-hexavinyldisiloxane, 1,1,1,3,3-pentavinylmethyldisiloxane, 1,1,1,3,3-n-octylpentamethyldisiloxane, 1,1,1,3,3-chloromethylpentamethyldisiloxane, 1,1,3,3-tetramethyl-1,3-diallyldisiloxane and 1,3-dimethyl-1,1,3-tetravinyldisiloxane. Among these, 1,1,1,3,3,3-hexamethyldisiloxane and 1,1,1,3,3,3-hexaphenyldisiloxane are preferred.

Examples of organosilicon compounds of general formula (14) include trimethylchlorosilane, triethylchlorosilane, ethyldimethylchlorosilane, trivinylchlorosilane, dimethylvinylchlorosilane, triphenylchlorosilane, dimethylphenylchlorosilane, methyldiphenylchlorosilane, trimethylmethoxysilane, trimethylethoxysilane, triethylmethoxysilane, triethylethoxysilane, triphenylmethoxysilane and triphenylethoxysilane. Among these, trimethylchlorosilane and trimethylethoxysilane are preferred.

Examples of organosilicon compounds containing a hydrosilyl group of general formula (15) include 1,1,3,3-tetramethyldisiloxane and 1,1,1,3,3-pentamethyldisiloxane. 1,1,3,3-tetramethyldisiloxane is particularly preferred.

In addition, in the general formulae (15) and (16), n satisfies the condition 1≤n≤3. In the general formula (15), the “n” associated with the H and the R¹ that are bonded to one silicone atom and the “n” associated with the H and the R¹ that are bonded to the other silicone atom may be the same or different.

Examples of organosilicon compounds containing a hydrosilyl group of general formula (16) include dimethylchlorosilane, diphenylchlorosilane, dimethylmethoxysilane and dimethylethoxysilane. Dimethylchlorosilane and dimethylmethoxysilane are particularly preferred.

Examples of hydrolysable silanes of general formula (17) include tetrachlorosilane, tetramethoxysilane and tetraethoxysilane. Examples of partial hydrolytic condensates of the hydrolysable silane include tetramethoxysilane condensates and tetraethoxysilane condensates. Examples of metal salts of the hydrolysable silane include water glass, sodium silicate, and potassium silicate. Tetraethoxysilane and tetraethoxysilane condensates are particularly preferred.

In this invention, to a mixture of one or more compounds chosen from the organosilicon compounds of general formulae (13) and (14), one or more compounds chosen from organosilicon compounds containing a hydrosilyl group of general formulae (15) and (16) and one or more compounds chosen from hydrolysable silanes of general formula (17), it is possible to add partial hydrolytic condensates of these hydrolysable silanes and metal salts of these hydrolysable silanes before hydrolysis under an acid catalyst, or a mixture of one or more compounds chosen from the organosilicon compounds of general formula (18) or of general formula (19) may be added after such a hydrolysis and before the rehydrolysis described below.

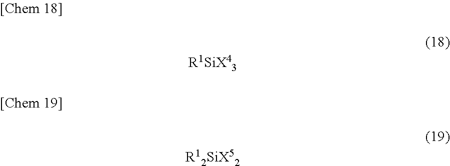

where
each R¹ is an identical or different alkyl, aryl or aralkyl group with 1 to 30 carbon atoms, or a group substituted by a halogen, a group substituted by an amino or a group substituted by a carboxyl of the latter;
X⁴ and X⁵ are hydrolysable functional groups.

In the general formulae (18) and (19), the examples and the preferred ranges for R¹ are the same as those mentioned above.

In the general formula (18), X⁴ is a hydrolysable functional group which is directly bonded to a silicon atom. Examples include halogen atoms such as chlorine and bromine atoms, alkoxy groups such as methoxy, ethoxy, propoxy and butoxy groups, alkenoxy groups, acyloxy groups, amide groups and oxime groups. Among these, from the point of view of availability and rate of hydrolysis, a methoxy group, an ethoxy group or a chlorine atom is preferred. The hydrolysable groups X⁴ on the same molecule may be similar or different.

In the general formula (19), X⁵ is a hydrolysable functional group which is directly bonded to a silicon atom. Examples include halogen atoms such as chlorine and bromine atoms, alkoxy groups such as methoxy, ethoxy, propoxy and butoxy groups, alkenoxy groups, acyloxy groups, amide groups and oxime groups. Among these, from the point of view of availability and rate of hydrolysis, a methoxy group, an ethoxy group or a chlorine atom is preferred. The hydrolysable groups X⁵ on the same molecule may be similar or different.

Examples of silicon compounds of general formula (18) include methyltrimethoxysilane, methyltriethoxysilane, ethyltrimethoxysilane, pentyltriethoxysilane, phenyltriethoxysilane, benzyltriethoxysilane, chloropropyltriethoxysilane, bromopropyltriethoxysilane, cyclohexyltrimethoxysilane, triopropyltrimethoxysilane and methyltrichlorosilane. Among these, methyltrimethoxysilane, methyltriethoxysilane and methyltrichlorosilane are preferred.

Examples of silicon compounds of general formula (19) include dimethyldimethoxysilane, dimethyldiethoxysilane, diethyldimethoxysilane, dipentyldiethoxysilane, diphenyldiethoxysilane, dibenzyldiethoxysilane, dichloropropyldiethoxysilane, dibromopropyldiethoxysilane, dicyclohexyldimethoxysilane, difluoropropyldimethoxysilane and dimethyldichlorosilane. Among these, dimethyldimethoxysilane, dimethyldiethoxysilane and dimethyldichlorosilane are preferred.

A specific example of a process for preparing the silicone resin containing a hydrosilyl group of that is used as raw material in the present invention is described. A solvent (in particular an organic solvent) and a hydrolysis raw material (a mixture of one or more compounds chosen from organosilicon compounds of general formulae (13) and (14), one or more compounds chosen from organosilicon compounds containing a hydrosilyl group of general formulae (15) and (16), and one or more compounds chosen from the hydrolysable silanes of general formula (17), the partial hydrolytic condensates of these hydrolysable silanes and the metal salts of these hydrolysable silanes) are loaded into a reactor, an acid is added as catalyst, and water is added dropwise with stirring. It is also possible in this case to add the organic solvent after the dropwise addition of the water has been completed. Since the hydrolysis is preferably carried out under acid conditions, the addition of an acid catalyst is essential.

The temperature during the dropwise addition of water is preferably between 0° C. and 80° C., and more preferentially between 0° C. and 50° C. By keeping the temperature within this range, the heat of reaction of the hydrolysis reaction on the hydrolysis starting product in the system can be kept low. The amount of water added dropwise, expressed as a molar ratio per mole of hydrolysable functional groups (alkoxy groups, etc.) is between 0.6 and 2, and preferably between 1.0 and 1.8. By keeping the amount of water added within this range, the deactivation of the hydrosilyl groups can be further suppressed.

In order to suppress a decrease in the reaction rate due to the retention and increase of the viscosity of the uniform reaction system during the hydrolysis reaction, it is preferable to use an organic solvent as solvent in the hydrolysis reaction. It is also desirable to use a solvent having a boiling point higher than the reflux temperature during the hydrolysis.

As examples of organic solvents, mention may be made of cyclic organopolysiloxanes such as octamethylcyclotetrasiloxane, decamethylcyclopentasiloxane and dodecamethylcyclohexasiloxane; aromatic hydrocarbons such as toluene and xylene; organic solvents of ketone type such as acetone, methyl ethyl ketone, diethyl ketone and methyl isobutyl ketone; and aliphatic hydrocarbons such as hexane, heptane, octane and cyclohexane.

In some cases, an alcoholic solvent having 1 to 10 carbon atoms may be used concomitantly. Examples include methanol, ethanol, 1-propanol, 2-propanol, 1-butanol, 2-methyl-1-propanol, 2-butanol, 2-methyl-2-propanol, 1-pentanol, 2-methylbutanol, 2-pentanol, 1-hexanol, 2-methylpentanol, 1-heptanol, 1-octanol, 1-nonanol, 1-decanol, phenol, benzyl alcohol, ethylene glycol and 1,2-propylene glycol. Since alcoholic solvents undergo alcohol exchange reactions with hydrolysable groups such as alkoxy groups, the use of a long-chain alcoholic solvent limits the rate of the hydrolysis reaction. Consequently, methanol, ethanol, 1-propanol and 2-propanol are particularly preferred.

The solvent used is included in an amount, relative to the overall reaction system, of from 1% to 80% (here and below, “%” refers to the percentage by weight), and in particular from 5% to 50%. Within this range, the reaction system remains uniform and the reaction takes place efficiently.

Examples of acid catalysts include hydrochloric acid, sulfuric acid, sulfurous acid, fuming sulfuric acid, oxalic acid, p-toluenesulfonic acid, methanesulfonic acid, trifluoromethanesulfonic acid, phosphoric acid, formic acid, acetic acid, propionic acid, benzoic acid and citric acid. The acid catalyst can be used in a small amount, an amount of the order of 0.001% to 10% of the overall reaction system being preferred.

After the water has been added dropwise as mentioned above, the hydrolysis reaction is carried out by heating the system to a temperature of between 50° C. and 150° C., preferably between 80° C. and 120° C., for approximately 2 to 8 hours. During this time, by carrying out the reaction at a temperature below the boiling point of the organic compound containing hydrosilyl groups used, the deactivation of the hydrosilyl groups can be further suppressed.

After having carried out the hydrolysis in this manner on the starting product of the above hydrolysis in the presence of an acid catalyst, the system is cooled to a temperature of between 10° C. and 100° C., preferably between 10° C. and 60° C., more preferably between 10° C. and 30° C., and even more preferably to 25° C.

After the above hydrolysis, the system is neutralized between 10° C. and 40° C. with a basic catalyst such as an alkali metal carbonate, an alkali metal bicarbonate or an alkali metal hydroxide. At this time, by using a strong basic catalyst and a weak basic catalyst together, the deactivation of the hydrosilyl group is suppressed and the condensation reaction of the organosilicon resin is further promoted. Among the examples of such highly basic catalysts, mention may be made of lithium hydroxide, sodium hydroxide, potassium hydroxide, calcium hydroxide and barium hydroxide. Examples of weakly basic catalysts include sodium carbonate, calcium carbonate and sodium bicarbonate. With respect to combinations of a strong basic catalyst with a weak basic catalyst, from the point of view of the ease of obtaining a high molecular weight, a combination of sodium hydroxide and calcium carbonate is desirable. With this combination, the molecular weight increases sufficiently, which makes it possible to more reliably obtain a high molecular weight organosilicon resin containing hydrosilyl groups.

The basic catalyst must be used in an amount greater than the molar equivalent of the acid catalyst. The fact of carrying out the neutralization with an amount of basic catalyst greater than the molar equivalent of the acid catalyst promotes the condensation reaction of the organosilicon resin, which results in an increase in the molecular weight and makes it possible to obtain a high molecular weight organosilicon resin containing hydrosilyl groups. The amount of basic catalyst used is preferably in the range of 1.0 to 3.0 molar equivalents of the acid catalyst. Adjusting the amount of addition within this range promotes the condensation reaction of the organosilicon resin containing hydrosilyl groups, which makes it possible to obtain a resin of target molecular weight.

After neutralization, the alcohols formed, the solvent and the excess water can be removed by heating between 95° C. and 120° C. under normal or reduced pressure. Then, after confirmation that the alcohols formed, the solvent and the excess water have been removed, the condensation reaction is carried out by heating between 120° C. and 150° C. for about 2 to 5 hours. An organosilicon resin containing a hydrosilyl group is thus obtained.

In the process described above for preparing a silicone resin containing a hydrosilyl group, the ratio of the combined molar amount of the compounds of general formulae (13), (14), (15) and (16) to the molar amount of SiO_{4/2} units in the compound of general formula (17), expressed as the molar ratio ((13)+(14)+(15)+(16)):(19) is preferably from 0.3:1 to 2:1, and more preferably from 0.6:1 to 1.3:1.

Moreover, the ratio of the combined molar amount of the compounds of general formulae (13) and (14) to the combined molar amount of the compounds of general formulae (15) and (16), expressed as the molar ratio ((13)+(14)):((15)+(16)), is preferably from 0.3:1.0 to 2.0:1.0, and more preferably from 0.6:1.0 to 1.3:1.0. By setting the values within these ranges, the amount of hydrosilyl groups included in the organosilicon resin containing hydrosilyl groups can be quantitatively varied more precisely. In the present invention, by thus varying the amounts in which the compounds of general formulae (15) and (16) are loaded, it is possible to vary quantitatively the amount of hydrosilyl groups included on the organosilicon resin.

In the process described above for preparing a silicone resin containing hydrosilyl groups, after having carried out the hydrolysis, in the presence of an acid catalyst, a mixture of one or more compounds chosen from organosilicon compounds of general formulae (13) and (14) with one or more compounds chosen from the hydrolysable silanes of general formula (17), partial hydrolytic condensates of these hydrolysable silanes and metal salts of these hydrolysable silanes, it is also possible to add gradually, dropwise, one or more compounds chosen from the organosilicon compounds containing a hydrosilyl group, of general formulae (15) and (16).

Next, a rehydrolysis is carried out. At this stage, the rehydrolysis reaction is preferably carried out by heating to a temperature below the boiling point of the silicone compound containing hydrosilyl groups, for example to a temperature preferably between 40° C. and 150° C., and more preferably between 40° C. and 120° C., for approximately 2 to 8 hours. When the reaction is carried out in this temperature range, the deactivation of the hydrosilyl groups can be further suppressed.

In the process for preparing the silicone resin containing hydrosilyl groups, the reaction of formula (20) below, in which some of the hydrosilyl groups are deactivated, can occur.

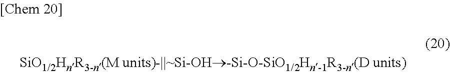

where R is a monovalent hydrocarbon group with 1 to 10 carbon atoms, and n′ is an integer from 1 to 3.

However, by suitably setting the order in which the raw materials are added, that is to say by hydrolysing a mixture of one or more compounds chosen from organosilicon compounds of general formulae (13) and (14) with one or more compounds chosen from hydrolysable silanes of general formula (17), partial hydrolytic condensates of these hydrolysable silanes and metal salts of these hydrolysable silanes, and by then adding one or more compounds chosen from the organosilicon compounds containing a hydrosilyl group of general formulae (15) and (16) and by carrying out a rehydrolysis, the above reaction (20) can be kept to a minimum. This reaction can be further suppressed by astutely modifying the amounts in which the raw materials are added and the type of catalyst used.

The amount of hydrosilyl groups included in the organosilicon resin containing hydrosilyl groups which is thus obtained is readily adjustable, and it is even possible to introduce a large amount of hydrosilyl groups by varying the amount of the organosilicon compound containing hydrosilyl groups which is loaded. Moreover, by varying the amount of hydrolysis starting materials used, the type and amount of acid catalyst added, the reaction temperature and time, the amount of solvent added and the method of addition, the molecular weight range, the shape and other characteristics of the organosilicon resin can be adjusted, which makes it possible to prepare an organosilicon resin containing hydrosilyl groups for the intended application.

The silicone resin containing a hydrosilyl group obtained as described above has the average formula (7) above and is composed of Q units (SiO_{4/2}) and M units ((R¹₃SiO_{1/2}) and (HₙR₃₋ₙSiO_{1/2})) as essential constituents, and also D units (R¹₂SiO_{2/2}) and T units (R¹SiO_{3/2}) as optional constituents. It may be in the form of a solid or a liquid at 25° C., although from the point of view of the formability of the film, it is preferably a solid. Among the examples, mention may be made of MQ resins, MTQ resins, MDQ resins and MDTQ resins. The weight-average molecular mass is preferably between 2000 and 30 000, although the range from 3000 to 15 000 is more preferred from the point of view of performance and ease of carrying out operations such as filtration. The weight-average molecular mass can be determined as the weight-average molecular mass equivalent to polystyrene in gel permeation chromatography (GPC).

### Process for Preparing the Glycerolated Silicone Resin

A specific example of a process for preparing the glycerolated silicone resin according to the invention is described below.

As mentioned above, the glycerolated silicone resin according to the invention can be obtained by the step of hydrosilylation:
(A) of a silicone resin containing a hydrosilyl group of average formula (7) below:

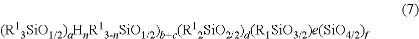

in which
   each R¹ is an identical or different alkyl, aryl or aralkyl group with 1 to 30 carbon atoms, or a group substituted by a halogen, a group substituted by an amino or a group substituted by a carboxyl of the latter;
   the subscripts a, b, c, d, e and f are integers that satisfy the conditions 0≤a≤400, 0<b≤200, 0≤c≤400, 0≤d≤320, 0≤e≤320, 0<f≤1000 and 0.5≤(a+b+c)/f≤1.5;
   n is an integer which satisfies the condition 1<n≤3, with
(B) one or more compounds which are chosen from the compounds terminated by an alkenyl group of general formulae (8), (9), (10), (11) and (12) below.

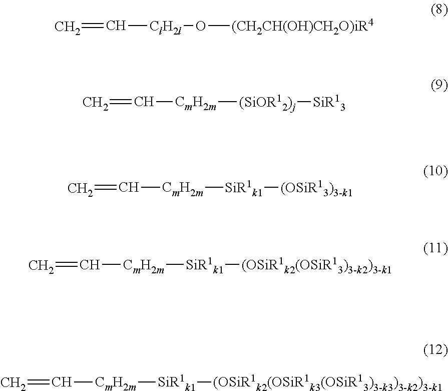

where
   R⁴ is a substituted or unsubstituted monovalent hydrocarbon group or a hydrogen atom,
   the subscripts l and i are integers that satisfy the conditions 0≤l≤15 and 0≤i≤5;
   the subscripts m, j and k₁ to k₃ are integers that satisfy the conditions 0≤m≤5, 0≤j≤500, 0≤k₁≤2, 0≤k₂≤2 and 0≤k₃≤2; said silicon resin comprises a compound of general formula (8).

The organosilicon resin containing hydrosilyl groups of average composition formula (7) and the compound having terminal alkenyl groups of general formula (8), (9), (10), (11) or (12) are mixed in a molar ratio, expressed as hydrosilyl groups/terminal unsaturated groups, which is preferably 0.5 to 2.0, and more preferentially from 0.8 to 1.2.

The addition reaction is preferably carried out in the presence of a platinum or rhodium catalyst. Specific examples include chloroplatinic acid, chloroplatinic acid modified by an alcohol, and chloroplatinic acid-vinylsiloxane complexes. When an excessive amount of catalyst is included, discoloration of the sample occurs, and thus the amount of platinum or rhodium is preferably 50 ppm or less, and more preferably 20 ppm or less.

Moreover, if necessary, the addition reaction can be carried out in the presence of an organic solvent. Among the examples of organic solvents, mention may be made of cyclic organopolysiloxanes such as octamethylcyclotetrasiloxane, decamethylcyclopentasiloxane and dodecamethylcyclohexasiloxane; aromatic hydrocarbons such as toluene and xylene; solvents of ketone type such as acetone, methyl ethyl ketone, diethyl ketone and methyl isobutyl ketone; aliphatic hydrocarbons such as hexane, heptane, octane and cyclohexane; and aliphatic alcohols such as methanol, ethanol, 1-propanol, 2-propanol, 1-butanol, 2-methyl-1-propanol, 2-butanol, 2-methyl-2-propanol, 1-pentanol, 2-methylbutanol, 2-pentanol, 1-hexanol, 2-methylpentanol, 1-heptanol, 1-octanol, 1-nonanol, 1-decanol, phenol, benzyl alcohol, ethylene glycol and 1,2-propylene glycol. From the point of view of reactivity, ethanol, 1-propanol and 2-propanol are preferred.

The amount of solvent used is preferably from 1% to 80%, and more preferably from 5% to 50%, of the overall reaction system. In the above range, the reaction system is kept uniform and the reaction takes place efficiently.

The conditions of the addition reaction are not particularly limited, although heating at reflux at a temperature of between 50° C. and 150° C., in particular between 80° C. and 120° C., for approximately 1 to 10 hours is preferred.

After the addition reaction, the step of removing the rhodium or platinum catalyst used with the activated carbon can be included. The amount of activated carbon used is preferably from 0.001% to 5.0%, and notably from 0.01% to 1.0%, of the overall system. By fixing the amount of activated carbon in this range, the discoloration of the sample can be better suppressed.

After the addition reaction, if necessary, the step of removing the remaining hydrosilyl groups can be included. In particular, in cases where use in applications such as cosmetic preparations is intended, there is a possibility that these hydrosilyl groups become deactivated over time due to dehydrogenation reactions, which poses a problem from the point of view of safety. It is therefore preferable to include a step of maintaining the hydrosilyl groups.

An example of a step for removing hydrosilyl groups is the process of hydrolysing the unreacted hydrosilyl groups by adding a basic catalyst such as an alkali metal carbonate, an alkali metal bicarbonate or an alkali metal hydroxide, followed by neutralization by the addition of an amount of acid catalyst equal to the molar equivalent of the basic catalyst. Specific examples of the basic catalyst include strong basic catalysts such as lithium hydroxide, sodium hydroxide, potassium hydroxide, calcium hydroxide, and barium hydroxide; and weak basic catalysts such as sodium carbonate, calcium carbonate, and sodium bicarbonate. From the point of view of promoting the dehydrogenation reaction, the use of a strong basic catalyst is particularly preferred, sodium hydroxide being particularly preferred. Among the acid catalysts, mention may be made of hydrochloric acid, sulfuric acid, sulfurous acid, fuming sulfuric acid, oxalic acid, p-toluenesulfonic acid, methanesulfonic acid, trifluoroacetic acid, trifluoromethanesulfonic acid, phosphoric acid, formic acid, acetic acid, propionic acid, benzoic acid and citric acid. In general, instead of using the acid or base alone, it is best to use them with water and heat them to a temperature not greater than the boiling point of water.

After the addition reaction, if necessary, a deodorization step for reducing the odour can be included. When use in applications such as cosmetic preparations in particular is intended, because the product acquires an odour over time, it is preferable to include a deodorization step. The mechanism for deodorizing common polyether-modified silicones can be explained as follows. When an addition reaction between a polyether etherified with allyl groups and a hydropolyorganosiloxane is carried out in the presence of a platinum catalyst, the allyl groups rearrange internally in the form of side reactions, forming a polyether etherified with propenyl groups. This propenyl-etherified polyether has no reactivity with the hydropolyorganosiloxane, and thus remains in the system as an impurity. It is believed that when water acts on this propenyl-etherified polyether, the propenyl ether hydrolyses, giving rise to propionaldehyde, which gives off an unpleasant odour. It is known that the above hydrolysis reaction is further promoted in the presence of an acid catalyst. Consequently, when the polyether-modified silicone is used in a water-based cosmetic preparation, due to oxidative deterioration of the polyether, the preparation tends to become acidic over time, promoting the hydrolysis reaction described above and giving rise to the appearance of a bad odour.

Typical examples of the deodorization step include two approaches. The first is the one in which, by adding an acid catalyst to the solution after the addition reaction, all the propenyl ether remaining in the system is hydrolysed and the propionaldehyde which forms is removed by strip purification (JP No. 2137062).

Specific examples of the acid catalyst used in the first approach include hydrochloric acid, sulfuric acid, sulfurous acid, fuming sulfuric acid, oxalic acid, p-toluenesulfonic acid, methanesulfonic acid, trifluoroacetic acid, trifluoromethanesulfonic acid, phosphoric acid, formic acid, acetic acid, propionic acid, benzoic acid and citric acid. These acids are used in combination with water. In cases where it is necessary to remove the acid which has been used, it is preferable to use an acid with a low boiling point, such as hydrochloric acid, formic acid, acetic acid or trifluoroacetic acid. Similarly, from the point of view of the effectiveness of the treatment, it is preferable to use a strong acid such as hydrochloric acid or trifluoroacetic acid.

The treatment temperature is preferably set at 80° C. or less in order to avoid oxidation of the hydrophilic groups. The amount of acidic aqueous solution added is preferably set at from 0.1% to 100% relative to the organosilicon resin modified with organic groups. The use of 5% to 30% is more preferred.

From the point of view of productivity, the process consisting in adding an aqueous solution to the post-reaction solution so as to adjust the pH to 7 or less and in carrying out a strip purification after stirring under heating is preferred. The purification of the strip can be carried out at normal temperature or under reduced pressure. The temperature conditions are preferably fixed at 120° C. or less. In order to efficiently purify the strip under these temperature conditions, it is preferable to carry out this operation under reduced pressure; when it is carried out at normal pressure, the operation is preferably carried out under a stream of inert gas, such as nitrogen or argon.

The second approach is that in which, by adding hydrogen to the solution after the addition reaction, the unsaturated double bonds are alkylated (subjected to a hydrogenation reaction) and the formation of propionaldehyde over time is controlled in a stable manner (U.S. Pat. No. 5,225,509; JP A H07-330907).

The hydrogenation reactions comprise methods involving the use of hydrogen and methods involving the use of metal hydrides, and there are also homogeneous reactions and heterogeneous reactions. These methods can be used alone but it is also possible to use them in combination. However, given the advantage that there is no trace of catalyst used in the product, a heterogeneous catalytic hydrogenation reaction using a solid catalyst is preferred.

The solid catalyst is, for example, nickel, palladium, platinum, rhodium, cobalt, chromium, copper, iron and others, in the uncombined form or in the compound form. In this case, it is not necessary to use a catalyst support. However, when a catalyst support is used, the support may be, for example, activated carbon, silica, silica-alumina, alumina or zeolite. These catalysts can be used alone, but it is also possible to use them in combination. The preferred catalyst is Raney nickel, which is economically advantageous. Since Raney nickel is generally developed and used with an alkali, it is necessary to carefully measure the pH of the reaction system. Moreover, the reaction system becomes weakly alkaline, which is particularly effective for deodorization when the hydrolysis reaction is carried out with an acidic aqueous solution.

It is preferable to carry out the hydrogenation reaction at a pressure generally between 1 and 100 MPa and between 50° C. and 200° C. The hydrogenation reaction can be carried out batchwise or continuously. In the case of a batch process, the reaction time depends, for example, on the amount of catalyst and on the temperature, but it is generally between 3 and 12 hours. The hydrogen pressure can be adjusted to an appropriate fixed pressure. The end point of the hydrogenation reaction is the point at which the hydrogen pressure has stopped changing, and it can therefore be determined by carefully monitoring a pressure gauge.

The amount of aldehyde included in the glycerolated silicone resin which has been purified by this acid treatment and this hydrogenation treatment can be set to 70 ppm or less, preferably to 20 ppm or less, and more preferably to 10 ppm or less.

It is also possible to combine the two types of deodorization steps mentioned above. In the approach which involves an acid treatment, decomposition and removal of the aldehyde compound is possible, but as there is a limit to the complete removal of the unsaturated double bonds, the formation of odorous aldehyde from this cannot be completely suppressed. In the approach which involves a hydrogenation reaction, by removing the unsaturated double bonds, it is possible to reduce the amount of aldehyde compound which is formed as a result of this. However, the aldehyde condensate which is formed with the condensation of a portion of the aldehyde remains in the system even after such a treatment has been carried out and the removal by strip purification is also difficult. Consequently, by alkylating the unsaturated double bonds which remain when the solution, following the addition reaction, is subjected to a hydrogenation, and by subsequently decomposing the aldehyde condensate in the system by adding an acid catalyst, complete deodorization is possible (WO2002/05588).

The weight-average molecular mass of the glycerolated silicone resin of average formula (I) preferably ranges from 1000 to 100 000; from the point of view of performance and ease of operations such as filtration, the weight-average molecular mass preferably varies from 3000 to 50 000. Here and below, the weight-average molecular weight can be determined as the weight-average molecular weight equivalent to polystyrene in gel permeation chromatography (GPC).

The glycerolated silicone resin according to the invention is in a form at 25° C. which may be solid or liquid; from the point of view of the formability of the film, it is preferably solid.

In particular, the glycerolated silicone resin according to the invention of formula (I) for which the subscripts b and c satisfy the conditions 00<b≤30 and 0≤c≤30, the subscript i in the general formula (2) is an integer which satisfies the condition 0<i≤3 and the subscript j in the general formula (3) satisfies the condition 0≤j≤10 is in the form of a solid at 25° C. and preferably has a weight-average molecular mass which preferably ranges from 1000 to 100 000 and more preferentially from 3000 to 50 000.

The glycerolated silicone resins according to the invention have a hydrophilic-lipophilic balance (HLB), as determined by Griffin's formula, preferably ranging from 0.1 to 15, and more preferably from 1.0 to 8.0.

According to a preferred form, the composition of the invention comprises at least one glycerolated silicone resin of formula (1) of (3-glyceroxypropyl) dimethylsiloxy trimethylsiloxysilicate type corresponding to formula (21) below:

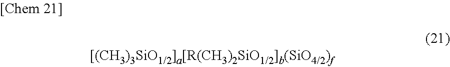

where
R denotes the 3-glyceroxypropyl group of structure
C₃H₆OCH₂—CH(OH)CH₂OH;
the subscripts a, b and f are integers that satisfy the conditions 0≤a≤400, 0<b≤30, 0<f≤1000 and 0.5≤(a+b)/f≤1.5.

According to a particularly preferred form, the glycerolated silicone resin of (3-glyceroxypropyl) dimethylsiloxy trimethylsiloxysilicate type of formula (21) is in the form of a solution in at least one volatile oil.

For the purposes of the invention, the term “volatile oil” refers to any oil that is capable of evaporating on contact with the skin in less than one hour, at room temperature and atmospheric pressure. The volatile oil is a volatile cosmetic compound, which is liquid at room temperature, notably having a non-zero vapour pressure, at room temperature and atmospheric pressure, notably having a vapour pressure ranging from 2.66 Pa to 40 000 Pa, in particular ranging from 2.66 Pa to 13 000 Pa and more particularly ranging from 2.66 Pa to 1300 Pa.

The volatile oil in accordance with the invention may be chosen from the group constituted of hydrocarbon oils, silicone oils, and mixtures thereof.

The term “hydrocarbon oil” is understood to mean an oil containing predominantly hydrogen and carbon atoms and optionally one or more functions chosen from hydroxyl, ester, ether and carboxyl functions.

Within the meaning of the present invention, the term “silicone oil” denotes an oil comprising at least one Si—O group, and more particularly an organopolysiloxane.

The volatile hydrocarbon-based oils that may be used in the compositions according to the invention may be chosen from branched C8-C16 alkanes.

Mention may notably be made of C8-C16 isoalkanes of petroleum origin (also known as isoparaffins), such as isododecane (also known as 2,2,4,4,6-pentamethylheptane), isodecane, isohexadecane and, for example, the oils sold under the Isopar® or Permethyl® trade names. Use will more preferentially be made of isododecane.

Mention may be made, as examples of volatile silicone oil which can be used in the invention, of volatile silicone oils, such as volatile linear or cyclic silicone oils, in particular those having a viscosity of 2 to 8 centistokes (2×10⁻⁶ to 8×10⁻⁶ m²/s), and containing in particular from 2 to 7 silicon atoms, these silicones optionally comprising alkyl or alkoxy groups containing from 1 to 10 carbon atoms. Mention may in particular be made, as volatile silicone oils which can be used in the invention, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and their mixtures. Use will more preferentially be made of decamethylcyclopentasiloxane (D5).

According to a particularly preferred form, the glycerolated silicone resin of (3-glyceroxypropyl) dimethylsiloxy trimethylsiloxysilicate type of formula (21) is in the form of a solution containing 49.5% by weight of active material in isododecane, for instance the product manufactured under the trade name X-25-9138A® by SHIN ETSU with a weight-average molecular mass of 11 000.

According to a preferred form, in order to improve the sebum resistance of the composition of the invention, the composition of the invention comprises at least one glycerolated silicone resin and at least one non-glycerolated silicone resin in a weight ratio of the amount of glycerolated silicone resin to the amount of non-glycerolated silicone resin of greater than or equal to 0.8, and more preferentially greater than or equal to 1.0.

### Volatile Hydrocarbon Oil

The composition according to the invention comprises an oily phase comprising at least one volatile hydrocarbon oil.

The term “oil” is understood to mean any fatty substance that is in liquid form at room temperature (25° C.) and at atmospheric pressure (760 mmHg or 105 Pa).

The term “oily phase” is understood to mean an organic phase that is liquid at room temperature (25° C.) and at atmospheric pressure and that is immiscible in water. It comprises at least one oil and any ingredient which is soluble or miscible in said phase.

The volatile hydrocarbon oils that can be used in the compositions according to the invention may be chosen from branched C8-C16 alkanes.

Mention may notably be made of C8-C16 isoalkanes of petroleum origin (also known as isoparaffins), such as isododecane (also known as 2,2,4,4,6-pentamethylheptane), isodecane, isohexadecane and, for example, the oils sold under the Isopar® or Permethyl® trade names.

Mention may also be made of branched C8-C16 esters such as isohexyl neopentanoate. Other volatile hydrocarbon oils, such as petroleum distillates, in particular those sold under the name Shell Solt® by Shell, can also be used.

The volatile hydrocarbon oils which can be used in the compositions according to the invention can be chosen from volatile linear alkanes comprising from 6 to 14 carbon atoms.

As examples of linear alkanes that are suitable for use in the invention, mention may be made of the alkanes described in the patent applications by the company Cognis WO 2007/068371 or WO 2008/155059 (mixtures of different alkanes differing by at least one carbon). These alkanes are obtained from fatty alcohols, which are themselves obtained from coconut kernel oil or palm oil.

As examples of linear C6-C14 alkanes that are suitable for use in the invention, mention may be made of n-hexane (C6), n-heptane (C7), n-octane (C8), n-nonane (C9), n-decane (C10), n-undecane (C11), n-dodecane (C12), n-tridecane (C13) and n-tetradecane (C14), and mixtures thereof.

Mention may notably be made of n-dodecane (C12) and n-tetradecane (C14) sold by Sasol under the references, respectively, Parafol 12-97® and Parafol 14-97®, and also mixtures thereof.

According to another embodiment, a mixture of n-dodecane and n-tetradecane is used. It is in particular possible to use the dodecane/tetradecane mixture in the 85/15 ratio by weight sold by Biosynthis under the reference Vegelight 1214®.

According to yet another embodiment, use is made of a mixture of volatile linear C9-C12 alkanes with the INCI name: C9-12 Alkane, such as the product sold by the company Biosynthis under the reference Vegelight Silk®.

According to yet another embodiment, use is made of a mixture of n-undecane (C11) and of n-tridecane (C13), such as those obtained in Examples 1 and 2 of application WO 2008/155059 from Cognis and such as that sold under the trade name Cetiol Ultimate® by BASF.

According to a particularly preferred embodiment, the volatile hydrocarbon oil is chosen from branched C8-C16 alkanes, and more particularly isododecane, the mixture of volatile linear C9-C12 alkanes and the mixture of n-undecane (C11) and n-tridecane (C13).

According to a particularly preferred embodiment, the composition of the invention comprises at least one volatile oil chosen from C8-C16 isoalkanes of petroleum origin (also called isoparaffins), in particular isododecane.

The volatile hydrocarbon oil(s) is (are) preferably present in the composition of the invention in contents of less than or equal to 80% by weight and preferably from 40% to 70% by weight relative to the total weight of said composition.

### Lipophilic Thickener

The composition according to the invention comprises at least one lipophilic thickener.

The term “lipophilic thickener” is understood to mean any molecule, liposoluble or lipodispersible in the oily phase of the composition, which is capable of increasing the viscosity of the composition.

As lipophilic thickener, at least one lipophilic clay will preferably be used.

Clay denotes a material based on hydrated silicates and/or aluminosilicates, of lamellar structure.

The clays may be natural or synthetic, and they are made lipophilic by treatment with an alkylammonium salt such as a C10 to C22 ammonium chloride, in particular stearalkonium chloride or distearyldimethylammonium chloride.

They may be chosen from bentonites, in particular bentonites, hectorites and montmorillonites, beidellites, saponites, nontronites, sepiolites, biotites, attapulgites, vermiculites and zeolites.

They are preferably chosen from hectorites and bentonites.

According to a particularly preferred form, use will be made of a lipophilic clay chosen from hydrophobically modified bentonites and hydrophobically modified hectorites, in particular that are modified with a C10 to C22 quaternary ammonium chloride, such as:
a bentonite modified with stearalkonium chloride, such as the commercial products sold under the name Claytone AF®, Garamite VT®, Tixogel® LG-M, Tixogel® MP 250 Tixogel® VZ and Tixogel® VZ-V XR, by the company BYK Additives Inc; or the commercial products sold under the name Viscogel® B3, Viscogel® B4, Viscogel® B7, Viscogel® B8, Viscogel® ED, Viscogel® GM, Viscogel® S4 and Viscogel® SD by the company Bentec S.P.A;
a bentonite modified with stearalkonium chloride in the presence of at least propylene carbonate and at least one oil, such as the commercial products Dub Velvet Gum® from the company Stéarinerie Dubois Fils, Miglyol Gel T® from the company Cremer Oleo, Tixogel® CGT 6030, Tixogel® DBA 6060, Tixogel® FTN, TIXOGEL® FTN 1564, Tixogel® IPM, Tixogel® LAN, Tixogel® LAN 1563 from the company BYK Additives Inc.;
a hectorite modified with distearyldimethylammonium chloride (INCI name: Disteardimonium Hectorite), for instance the product sold under the name Bentone® 38VCG Rheological Additive by the company Elementis Specialties;
a hectorite modified with distearyldimethylammonium chloride in the presence of at least propylene carbonate or triethyl citrate and of at least one oil, such as the commercial products sold under the name Bentone® Gel DOA V, Bentone® Gel EUG V, Bentone® Gel IHD V, Bentone® Gel ISD V, Bentone® Gel MIO V®, Bentone® Gel PTM VO, Bentone® SS-71 V, Bentone® VS-5 PC V or Bentone® VS-5 by the company Elementis Specialities; the commercial products sold under the name Creagel® Bentone CPS/Hectone CPS or Creagel Bentone® ID/Hectone ID by the company Créations Couleurs; the commercial products sold under the name NS Gel DM1®, NS Gel PTIS® or NS MGel 1152® by the company Next Step Laboratories Stop.

More particularly, use will be made of a hectorite modified with distearyldimethylammonium chloride (INCI name: Disteardimonium Hectorite), for instance the product sold under the name Bentone® 38VCG Rheological Additive by the company Elementis Specialties.

The lipophilic thickener(s) can be present in the composition at concentrations ranging preferably from 0.5% to 10% by weight and more preferentially from 1% to 6% by weight, relative to the total weight of the composition.

### Silicone Polyamide

According to one particular form, the composition according to the invention also comprises at least one silicone polyamide.

The silicone polyamides are preferably solid at room temperature (25° C.) and atmospheric pressure (760 mmHg).

For the purposes of the invention, the term “polymer” means a compound containing at least two repeating units, preferably at least three repeating units and better still ten repeating units.

The silicone polyamides of the composition of the invention may be polymers of the polyorganosiloxane type, for instance those described in U.S. Pat. Nos. 5,874,069, 5,919,441, 6,051,216 and 5,981,680. According to the invention, the silicone polymers may belong to the following two families:
(1) polyorganosiloxanes comprising at least two amide groups, these two groups being located in the polymer chain, and/or
(2) polyorganosiloxanes comprising at least two amide groups, these two groups being located on grafts or branches.

According to a first variant, the silicone polymers are polyorganosiloxanes as defined above in which the units capable of establishing hydrogen interactions are located in the polymer chain.

The silicone polymers may be more particularly polymers comprising at least one unit corresponding to the general formula (I):

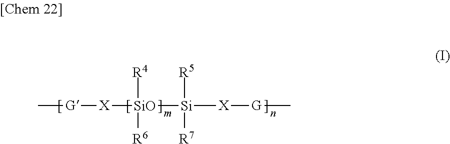

in which: G′ represents C(O) when G represents —C(O)—NH—Y—NH—, and G′ represents —NH— when G represents —NH—C(O)—Y—C(O)—,
R⁴, R¹, R⁶ and R⁷, which may be identical or different, represent a group chosen from:
   saturated or unsaturated, linear, branched or cyclic C1 to C40 hydrocarbon groups which may contain in their chain one or more oxygen, sulfur and/or nitrogen atoms, and which may be partially or totally substituted with fluorine atoms,
   C6 to C10 aryl groups, optionally substituted with one or more C1 to C4 alkyl groups,
   polyorganosiloxane chains optionally containing one or more oxygen, sulfur and/or nitrogen atoms,
the X groups, which may be identical or different, represent a linear or branched C1 to C30 alkylenediyl group, which may contain in its chain one or more oxygen and/or nitrogen atoms,
Y is a saturated or unsaturated C1 to C50 linear or branched alkylene, arylene, cycloalkylene, alkylarylene or arylalkylene divalent group, which may include one or more oxygen, sulfur and/or nitrogen atoms, and/or which may bear as substituent one of the following atoms or groups of atoms: fluorine, hydroxyl, C3 to C8 cycloalkyl, C1 to C40 alkyl, C5 to C10 aryl, phenyl optionally substituted with one to three C1 to C3 alkyl, C1 to C3 hydroxyalkyl and C1 to C6 aminoalkyl groups, or Y represents a group corresponding to formula (23):

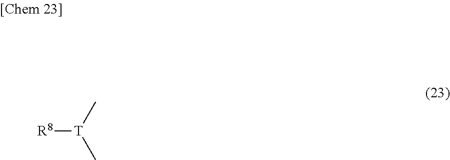

in which
T represents a linear or branched, saturated or unsaturated, C3 to C24 trivalent or tetravalent hydrocarbon group optionally substituted with a polyorganosiloxane chain, and which may contain one or more atoms chosen from O, N and S, or T represents a trivalent atom chosen from N, P and Al, and
R⁸ represents a linear or branched C1 to C50 alkyl group or a polyorganosiloxane chain, which may comprise one or more ester, amide, urethane, thiocarbamate, urea, thiourea and/or sulfonamide groups, which may optionally be bonded to another chain of the polymer,
n is an integer ranging from 2 to 500, preferably from 2 to 200, and m is an integer ranging from 1 to 1000, preferably from 1 to 700 and better still from 6 to 200. Preferably, m is an integer ranging from 50 to 150.

According to one embodiment of the invention, 80% of the groups R⁴, R⁵, R⁶ and R⁷ of the polymer are preferably chosen from methyl, ethyl, phenyl and 3,3,3-trifluoropropyl groups. According to another embodiment, 80% of the groups R⁴, R⁵, R⁶ and R⁷ of the polymer are methyl groups.

According to the invention, Y may represent various divalent groups, furthermore optionally including one or two free valencies to establish bonds with other units of the polymer or copolymer. Preferably, Y represents a group chosen from:
linear C1 to C20 and preferably C1 to C10 alkylene groups,
C30 to C56 branched alkylene groups which may comprise rings and unconjugated unsaturations,
C5-C6 cycloalkylene groups,
phenylene groups optionally substituted with one or more C1 to C40 alkyl groups,
C1 to C20 alkylene groups comprising from 1 to 5 amide groups,
C1 to C20 alkylene groups comprising one or more substituents, chosen from hydroxyl, C3 to C8 cycloalkane, C1 to C3 hydroxyalkyl and C1 to C6 alkylamine groups,
polyorganosiloxane chains of formula (24) or (25):

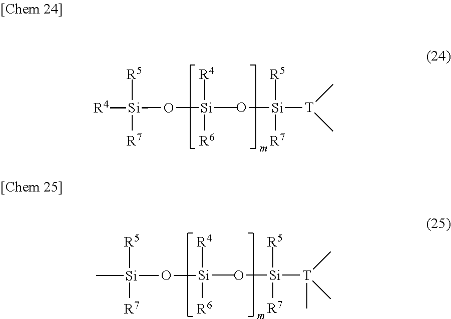

in which R⁴, R⁵, R⁶, R⁷, T and m are as defined above.

According to the second variant, the polyorganosiloxanes may be polymers comprising at least one unit corresponding to formula (II):

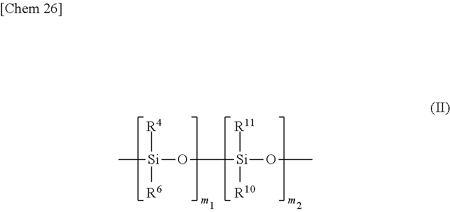

in which
R⁴ and R⁶, which are identical or different, are as defined above for formula (I),
R¹⁰ represents a group as defined above for R⁴ and R⁶, or represents the group of formula —X-G-R¹² in which X and G are as defined above for formula (I) and R¹² represents a hydrogen atom or a linear, branched or cyclic, saturated or unsaturated, C1 to C50 hydrocarbon group optionally comprising in its chain one or more atoms chosen from O, S and N, optionally substituted with one or more fluorine atoms and/or one or more hydroxyl groups, or a phenyl group optionally substituted with one or more C1-C4 alkyl groups,
R¹¹ represents the group of formula —X-G-R¹² in which X, G and R¹² are as defined above,
m1 is an integer ranging from 1 to 998, and
m2 is an integer ranging from 2 to 500.

According to a particular embodiment of the invention, the silicone polyamide may be a homopolymer, that is to say a polymer including several identical units, in particular units of formula (I) or of formula (II).

According to another particular embodiment of the invention, it is also possible to use a silicone polyamide formed from a copolymer including several different units of formula (I), that is to say a polymer in which at least one from among R⁴, R⁵, R⁶, R⁷, X, G, Y, m and n is different in one of the units. The copolymer may also be formed from several units of formula (II), in which at least one from among R⁴, R6, R¹⁰, R¹¹, m₁ and m₂ is different in at least one of the units.

It is also possible to use a polymer including at least one unit of formula (I) and at least one unit of formula (II), the units of formula (I) and the units of formula (II) possibly being identical to or different than each other.

According to one variant of the invention, it is also possible to use a polymer furthermore comprising at least one hydrocarbon-based unit including two groups that are capable of establishing hydrogen interactions, chosen from ester, amide, sulfonamide, carbamate, thiocarbamate, urea, urethane, thiourea, oxamido, guanidino and biguanidino groups, and combinations thereof.

These copolymers may be block polymers or grafted polymers.

According to an advantageous embodiment of the invention, the groups that are capable of establishing hydrogen interactions are amide groups of formulae —C(O)NH and —HNC(O).

In this case, the film-forming agent may be a polymer comprising at least one unit of formula (III) or (IV):

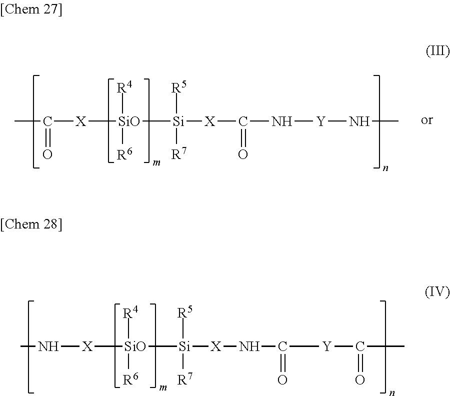

in which R⁴, R⁵, R⁶, R⁷, X, Y, m and n are as defined above.

In these polyamides of formula (III) or (IV), m ranges from 1 to 700, in particular from 15 to 500 and notably from 50 to 200, and n ranges in particular from 1 to 500, preferably from 1 to 100 and better still from 4 to 25; and
X is preferably a linear or branched alkylene chain having from 1 to 30 carbon atoms, in particular 1 to 20 carbon atoms, notably from 5 to 15 carbon atoms and more particularly 10 carbon atoms, and
Y is preferably an alkylene chain that is linear or branched, or which may comprise rings and/or unsaturations, containing from 1 to 40 carbon atoms, in particular 1 to 20 carbon atoms and better still from 2 to 6 carbon atoms, in particular 6 carbon atoms.

In formulae (III) and (IV), the alkylene group representing X or Y may optionally contain in its alkylene part at least one of the following components:
1 to 5 amide, urea, urethane or carbamate groups,
a C5 or C6 cycloalkyl group, and
a phenylene group optionally substituted with 1 to 3 identical or different C1 to C3 alkyl groups.

In formulae (III) and (IV), the alkylene groups may also be substituted with at least one component chosen from the group consisting of:
a hydroxyl group,
a C3 to C8 cycloalkyl group,
one to three C1 to C40 alkyl groups,
a phenyl group optionally substituted with one to three C1 to C3 alkyl groups,
a C1 to C3 hydroxyalkyl group, and
a C1 to C6 aminoalkyl group.

In these formulae (III) and (IV), Y may also represent a group of formula (29):

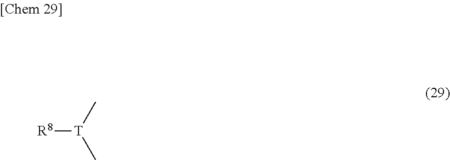

where R⁸ represents a polyorganosiloxane chain and T represents a group of formula (30):

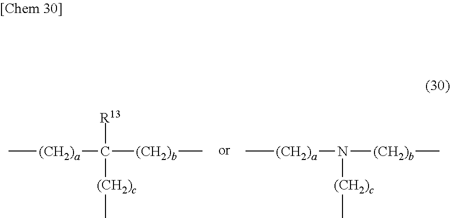

in which a, b and c are, independently, integers ranging from 1 to 10, and R¹³ is a hydrogen atom or a group such as those defined for R⁴, R⁵, R⁶ and R⁷.

In formulae (III) and (IV), R⁴, R⁵, R⁶ and R⁷ preferably represent, independently, a linear or branched C1 to C40 alkyl group, preferably a CH₃, C₂H₅, n-C₃H₇ or isopropyl group, a polyorganosiloxane chain or a phenyl group optionally substituted with one to three methyl or ethyl groups.

According to a preferred embodiment, the silicone polyamide comprises at least one unit of formula (III) or (IV).

As has been seen previously, the polymer may comprise identical or different units of formula (III) or (IV).

Thus, the polymer may be a polyamide containing several units of formula (III) or (IV) of different lengths, i.e. a polyamide corresponding to formula (V):

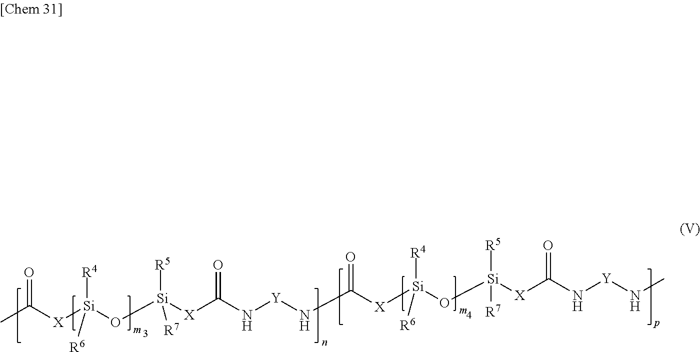

in which X, Y, n and R⁴ to R⁷ have the meanings given above, m₃ and m₄, which are different, are chosen in the range from 1 to 1000, and p is an integer ranging from 2 to 300. In this formula, the units may be structured to form either a block copolymer, or a random copolymer or an alternating copolymer.

In this copolymer, the units may be not only of different lengths, but also of different chemical structures, for example containing different groups Y. In this case, the polymer may correspond to formula (VI):

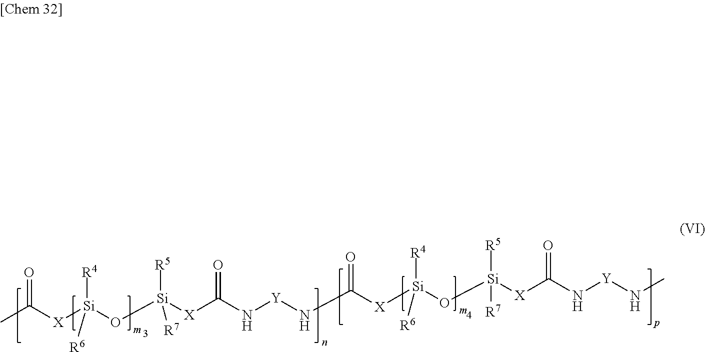

in which R⁴ to R⁷, X, Y, m₃, m₄, n and p have the meanings given above and Y1 is different from Y but is chosen from the groups defined for Y.

As previously, the various units may be structured to form either a block copolymer, or a random copolymer or an alternating copolymer.

In this first embodiment of the invention, the film-forming agent may also be constituted by a graft copolymer. Thus, the polyamide containing silicone units may be grafted and optionally crosslinked with silicone chains containing amide groups. Such polymers may be synthesized with trifunctional amines. In this case, the polymer may comprise at least one unit of formula (VII):

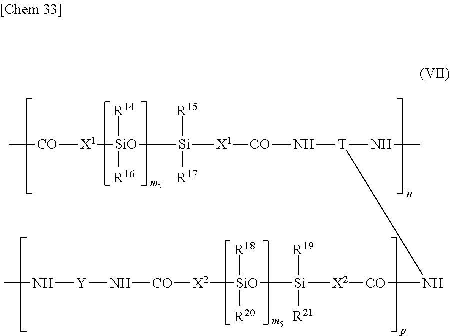

in which X¹ and X², which are identical or different, have the meaning given for X in formula (I), n is as defined in formula (I), Y and T are as defined in formula (I), R¹⁴ to R²¹ are groups chosen from the same group as R⁴ to R⁷, m₅ and m₆ are numbers in the range from 1 to 1000, and p is an integer ranging from 2 to 500.

In formula (VII), it is preferred that:
p is in the range from 1 to 25 and better still from 1 to 7,
R¹⁴ to R²¹ are methyl groups,
T corresponds to one of the following formulae:

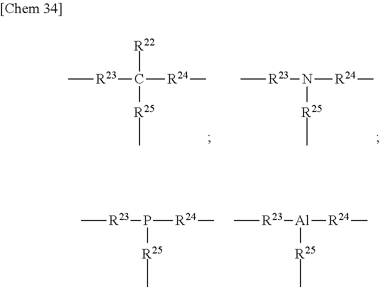

in which R²² is a hydrogen atom or a group chosen from the groups defined for R⁴ to R⁷, and R²³, R²⁴ and R²⁵ are, independently, linear or branched alkylene groups, and more preferably corresponds to the formula:

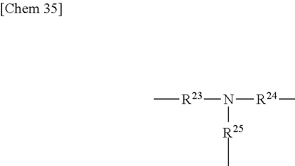

in particular with R₂₃, R₂₄ and R₂₅ representing —CH₂—CH₂—,
m₁ and m₂ range from 15 to 500 and better still from 15 to 45,
X¹ and X² represent —(CH₂)₁₀—, and
Y represents —CH₂—.

These polyamides containing a grafted silicone unit of formula (VII) may be copolymerized with silicone polyamides of formula (II) to form block copolymers, alternating copolymers or random copolymers. The weight percentage of grafted silicone units (VII) in the copolymer may range from 0.5% to 30% by weight.

According to the invention, as has been seen previously, the siloxane units may be in the main chain or backbone of the polymer, but they may also be present in grafted or pendent chains. In the main chain, the siloxane units may be in the form of segments as described above. In the pendent or grafted chains, the siloxane units may appear individually or in segments.

According to one embodiment variant of the invention, a copolymer of silicone polyamide and of hydrocarbon-based polyamide, or a copolymer including units of formula (III) or (IV) and hydrocarbon-based polyamide units, may be used. In this case, the silicone polyamide units may be located at the ends of the hydrocarbon-based polyamide.

According to a preferred embodiment, the silicone polyamide comprises units of formula (III).

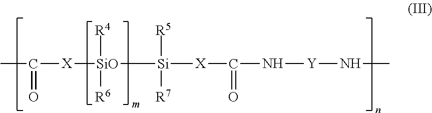

in which R⁴, R⁵, R⁶ and R⁷ represent, independently, a linear or branched C1 to C40 alkyl group, preferably a CH₃, C₂H₅, n-C₃H₇ or isopropyl group, a polyorganosiloxane chain or a phenyl group optionally substituted with one to three methyl or ethyl groups, and m ranges from 1 to 700, in particular from 15 to 500 and notably from 50 to 200 and n ranges in particular from 1 to 500, preferably from 1 to 100 and better still from 4 to 25.

Preferably, according to this embodiment, the groups R⁴, R⁵, R⁶ and R⁷ represent methyl groups, one from among X and Y represents an alkylene group of 6 carbon atoms and the other represents an alkylene group of 11 carbon atoms, n representing the degree of polymerization (DP) of the polymer.

As examples of such silicone polyamides, mention may be made of the compounds sold by the company Dow Corning under the names Dowsil 2-8179 Gellant® (DP 100) and Dowsil 2-8178 Gellant® (DP 15), the INCI name of which is Nylon-611/dimethicone copolymer.

Advantageously, the composition according to the invention comprises at least one polydimethylsiloxane block polymer of general formula (I) having a subscript m with a value of about 100.

The subscript m corresponds to the degree of polymerization of the silicone portion of the polymer.

More preferably, the composition according to the invention comprises at least one polymer comprising at least one unit of formula (III) in which m ranges from 50 to 200, in particular from 75 to 150 and is preferably about 100.

As examples of silicone polymers that may be used, mention may be made of one of the silicone polyamides obtained in accordance with Examples 1 to 3 of U.S. Pat. No. 5,981,680.

According to a preferred embodiment, use is made of a silicone polyamide polymer with the INCI name: Nylon-611/dimethicone copolymer sold by the company Dow Corning under the name Dowsil 2-8179 Gellant® (DP 100).

The polymers and/or copolymers used in the composition of the invention advantageously have a solid state to liquid state transition temperature ranging from 45° C. to 190° C. Preferably, they have a solid state to liquid state transition temperature ranging from 70° C. to 130° C. and better still from 80° C. to 105° C.

The content of silicone polyamide, expressed as active material, preferably ranges from 5% to 30% by weight, more preferentially from 10% to 25% by weight and more particularly from 8% to 15% by weight relative to the weight of the composition.

### Cosmetic Additives

The composition may contain conventional cosmetic additives such as colorants, preservatives, fragrances, antioxidants, moisturizers, lipophilic active agents such as vitamins, lipophilic UV-screening agents, fillers.

Of course, a person skilled in the art will take care to choose the optional additional additives and/or the amount thereof such that the advantageous properties of the composition according to the invention are not, or are not substantially, adversely affected by the envisaged addition.

### Colorants

The composition according to the invention may further comprise at least one colorant.

According to a particular form of the invention, the colorant may be chosen from pulverulent colorants, liposoluble dyes, and mixtures thereof.

### a) Pulverulent Colorants

The pulverulent colorants may be chosen from mineral pigments, organic pigments, pearlescent agents and mixtures thereof.

The term “pigments” means white or coloured, mineral or organic particles, which are insoluble in an aqueous medium, and which are intended to colour and/or opacify the resulting composition and/or deposit. These pigments may be white or coloured, and mineral and/or organic.

According to a particular embodiment, the pigments used according to the invention are chosen from mineral pigments.

The term “mineral pigment” refers to any pigment that satisfies the definition in Ullmann's encyclopaedia in the chapter on inorganic pigments. Among the mineral pigments that are useful in the present invention, mention may be made of zirconium oxide or cerium oxide, and also zinc oxide, iron oxide (black, yellow or red) or chromium oxide, manganese violet, ultramarine blue, chromium hydrate and ferric blue, titanium dioxide, and metal powders, for instance aluminium powder and copper powder. The following mineral pigments may also be used: Ta₂O₅, Ti₃O₅, Ti₂O₃, TiO, ZrO₂ as a mixture with TiO₂, ZrO₂, Nb₂O₅, CeO₂ or ZnS.

The size of the pigment of use in the context of the present invention is generally greater than 100 nm and can range up to 10 μm, preferably from 200 nm to 5 μm and more preferentially from 300 nm to 1 μm.

According to a particular form of the invention, the pigments exhibit a size characterized by a D[50] of greater than 100 nm and which can range up to 10 μm, preferably from 200 nm to 5 μm and more preferentially from 300 nm to 1 μm.

The sizes are measured by static light scattering using a commercial MasterSizer 3000® particle size analyser from Malvern, which makes it possible to determine the particle size distribution of all of the particles over a wide range which may extend from 0.01 μm to 1000 μm. The data are processed on the basis of the standard Mie scattering theory. This theory is the most suitable for size distributions ranging from submicronic to multimicronic; it makes it possible to determine an “effective” particle diameter. This theory is notably described in the publication by Van de Hulst, H. C., Light Scattering by Small Particles, Chapters 9 and 10, Wiley, New York, 1957.

D[50] represents the maximum size exhibited by 50% by volume of the particles.

According to a particular form of the invention, the mineral pigment comprises a lipophilic or hydrophobic coating, said coating preferably being present in the oily phase of the composition according to the invention.

According to a particular embodiment of the invention, the pigments may be coated according to the invention with at least one compound chosen from metal soaps; N-acylamino acids or salts thereof; lecithin and derivatives thereof; isopropyl triisostearyl titanate; isostearyl sebacate; natural plant or animal waxes; polar synthetic waxes; fatty esters; phospholipids; and mixtures thereof.

According to a particular embodiment, the pigments may be coated according to the invention with an N-acylamino acid or a salt thereof, which may comprise an acyl group containing from 8 to 22 carbon atoms, for instance a 2-ethylhexanoyl, caproyl, lauroyl, myristoyl, palmitoyl, stearoyl or cocoyl group.

The amino acid may be, for example, lysine, glutamic acid or alanine. The salts of these compounds may be the aluminium, magnesium, calcium, zirconium, zinc, sodium or potassium salts. Thus, according to a particularly preferred embodiment, the pigments may be coated with an N-acylamino acid derivative which may notably be a glutamic acid derivative and/or a salt thereof, and more particularly a stearoyl glutamate, for instance aluminium stearoyl glutamate. As examples of pigments treated with aluminium stearoyl glutamate, mention may be made of titanium dioxide pigments and black, red and yellow iron oxide pigments sold under the trade name NAI® by the company Miyoshi Kasei.

According to a preferred embodiment, the pigments according to the invention can be coated with isopropyl triisostearyl titanate. As examples of isopropyl titanium triisostearate (ITT)-treated pigments, mention may be made of titanium dioxide pigments and the black, red and yellow iron oxides sold under the trade names BWBO-I2® (iron oxide CI77499 and isopropyl titanium triisostearate), BWYO-I2® (iron oxide CI77492 and isopropyl titanium triisostearate) and BWRO-I2® (iron oxide CI77491 and isopropyl titanium triisostearate) by the company Kobo.

Among the mineral pigments, mention may also be made of pearlescent agents. They may be chosen from white pearlescent pigments such as mica coated with titanium or with bismuth oxychloride, coloured pearlescent pigments such as titanium mica with iron oxides, titanium mica notably with ferric blue or chromium oxide, titanium mica with an organic pigment of the abovementioned type, and also pearlescent pigments based on bismuth oxychloride.

The pigments that may be used according to the invention may also be organic pigments.

“Organic pigment” means any pigment that satisfies the definition in Ullmann's Encyclopedia in the chapter on organic pigments. The organic pigment may notably be chosen from nitroso, nitro, azo, xanthene, quinoline, anthraquinone, phthalocyanine, metal-complex type, isoindolinone, isoindoline, quinacridone, perinone, perylene, diketopyrrolopyrrole, thioindigo, dioxazine, triphenylmethane or quinophthalone compounds.

The organic pigment(s) may be chosen, for example, from carmine, carbon black, aniline black, melanin, azo yellow, quinacridone, phthalocyanine blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenol derivatives as described in patent FR 2 679 771.

These pigments may also be in the form of composite pigments as described in patent EP 1 184 426. These composite pigments may notably be composed of particles including a mineral core at least partially covered with an organic pigment and at least one binder for fixing the organic pigments to the core.

The pigment may also be a lake. The term “lake” means insolubilized dyes adsorbed onto insoluble particles, the assembly thus obtained remaining insoluble during use.

The inorganic substrates onto which the dyes are adsorbed are, for example, alumina, silica, calcium sodium borosilicate or calcium aluminium borosilicate and aluminium.

Among the organic dyes, mention may be made of cochineal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 1 O (CI 77 002), D&C Green 3 (CI 42 053) or D&C Blue 1 (CI 42 090).

Mention may be made, by way of an example of a lake, of the product known under the name D&C Red 7 (CI 15 850:1).

Preferably, the pulverulent colorant(s) is (are) preferably present in the composition in a content of less than or equal to 50% by weight, preferably from 25% to 40% by weight, more particularly from 3% to 15% by weight, relative to the total weight of the composition.

### b) Liposoluble Colorants

A composition according to the invention may comprise at least one liposoluble colorant, preferably in a proportion of at least 0.01% by weight relative to the total weight of the composition.

For obvious reasons, this amount is liable to vary significantly with regard to the intensity of the desired colour effect and of the colour intensity afforded by the colorants under consideration, and its adjustment clearly falls within the competence of a person skilled in the art.

For the purposes of the invention, the term “liposoluble colorant” means any natural or synthetic, generally organic compound, which is soluble in an oily phase or in solvents that are miscible with a fatty substance, and which is capable of imparting colour.

As liposoluble dyes that are suitable for use in the invention, mention may notably be made of synthetic or natural liposoluble dyes, for instance DC Red 17, DC Red 21, DC Red 27, DC Green 6, DC Yellow 11, DC Violet 2, DC Orange 5, Sudan red, carotenes (β-carotene, lycopene), xanthophylls (capsanthin, capsorubin, lutein), palm oil, Sudan brown, quinoline yellow, annatto and curcumin.

Preferably, the composition according to the invention comprises at least one pulverulent colorant of mineral pigment type, in particular chosen from metal oxides, and more particularly chosen from coated or uncoated titanium dioxides or black, red or yellow iron oxides, and mixtures thereof.

According to a particularly preferred embodiment, the composition according to the invention comprises at least one pulverulent colorant chosen from titanium dioxides according to the invention coated with one of isopropyl triisostearyl titanate, black, red or yellow iron oxides coated with isopropyl triisostearyl titanate, and mixtures thereof.

### Cosmetic Applications

The composition used according to the invention may be a composition for caring for and/or making up keratin materials, in particular eyebrows including the eyebrow hairs, the skin for implantation of said hairs and the contours thereof.

According to a particularly preferred form, the composition of the invention is anhydrous.

For the purposes of the invention, the expression “anhydrous composition” denotes, respectively, a composition which contains less than 5% by weight of water, preferably less than 2% by weight of water, indeed even less than 0.5% of water, relative to its total weight, and in particular a composition which is free of water.

More especially, the composition according to the invention is an eyebrow care and/or makeup product such as a mascara.

Such compositions are in particular prepared according to the general knowledge of a person skilled in the art.

### Packaging and Application Assembly or Kit

The present invention also relates to an assembly, or kit, for packaging and applying a cosmetic composition for coating keratin materials, comprising:
a packaging device comprising said cosmetic composition for coating keratin materials, as described above,
an applicator for said composition.

According to another aspect, the invention also relates to a makeup assembly comprising:
i) an applicator
ii) a composition in accordance with the invention placed inside a container.

The container can delimit one or more compartment(s). The container can, for example, be in the form of a tube.

Such an applicator can be integral with a cap reversibly fitted to said container between a position of closure of said container and a makeup position.

In an alternative form, such an applicator can be irreversibly fitted to said container. Mention may be made, as examples of applicators, of those of felt or brush type which can be constituted of synthetic fibers.

It is understood that, in the context of the present invention, the percentages by weight given for a compound or a family of compounds are always expressed by weight with respect to the total weight of the composition.

Throughout the patent application, the term “comprises one” should be understood as meaning “comprising at least one”, unless otherwise specified.

It is understood that the examples which follow are present by way of illustration and that they do not in any way limit the scope of the protection conferred by the present patent application.

### PREPARATION EXAMPLES

### Example 1: Preparation of a Decamethylcyclopentasiloxane Solution Containing 60% of Silicone Resin Modified with 3-Glyceroxypropyl Groups

A reactor was loaded with 1300 g of a decamethylcyclopentasiloxane solution containing 50% of a powdered organosilicon resin containing hydrosilyl groups, having a formula of average composition (E4) (weight average molecular weight, 4480; amount of hydrogen gas evolved, 8.0 ml/g), 30.7 g of glycerol monoallyl ether of formula (E5), 1300 g of 2-propanol and 0.7 g of a 0.5% solution of chloroplatinic acid in 2-propanol, and the reaction was carried out by heating for 6 hours at 100° C. The solvent was then removed by heating under reduced pressure. Next, 325 g of ethanol were added, after which 6.5 g of a 5% aqueous sodium hydroxide solution were added, thus hydrolysing the unreacted hydrosilyl groups, after which neutralization was carried out by adding 0.8 g of concentrated hydrochloric acid. After neutralization, 195 g of 0.01 N aqueous hydrochloric acid were added, thus hydrolysing the unreacted allyl ether groups on the polyoxyalkylene, and neutralization was carried out with 3.3 g of 5% aqueous sodium bicarbonate. The reaction product was heated under reduced pressure in order to remove the solvent and filtration was carried out, giving a decamethylcyclopentasiloxane solution of the silicone resin modified with 3-glyceroxypropyl groups of formula (E6). The solution had a clear and colourless appearance.

The decamethylcyclopentasiloxane solution of this silicone resin modified with 3-glyceroxypropyl groups was heated to 120° C. to 130° C. under reduced pressure in order to remove the decamethylcyclopentasiloxane. The product thus obtained was a solid powder which had an HLB of 0.9.

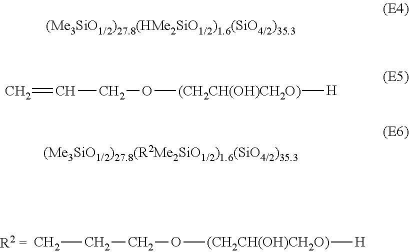

### Example 2: Preparation of an Isododecane Solution Containing 60 wt % of a Silicone Resin Modified with 3-Glyceroxypropyl Groups

A reactor was loaded with 1300 g of an isododecane solution containing 50% of a powdered organosilicon resin containing a hydrosilyl group, having a formula of average composition (E4) (weight average molecular weight, 4480; amount of hydrogen gas evolved, 8.0 ml/g), 30.7 g of glycerol monoallyl ether of formula (E5), 1300 g of 2-propanol and 0.7 g of a 0.5% solution of chloroplatinic acid in 2-propanol, and the reaction was carried out by heating for 6 hours at 100° C. The solvent was then removed by heating under reduced pressure. Next, 325 g of ethanol were added, after which 6.5 g of a 5% aqueous sodium hydroxide solution were added, thus hydrolysing the unreacted hydrosilyl groups, after which neutralization was carried out by adding 0.8 g of concentrated hydrochloric acid. After neutralization, 195 g of 0.01 N aqueous hydrochloric acid were added, thus hydrolysing the unreacted allyl ether groups on the polyoxyalkylene, and neutralization was carried out with 3.3 g of 5% aqueous sodium bicarbonate. The reaction product was then heated under reduced pressure in order to remove the solvent and filtration was carried out, thus giving an isododecane solution of the silicone resin modified with 3-glyceroxypropyl groups of formula (E6).

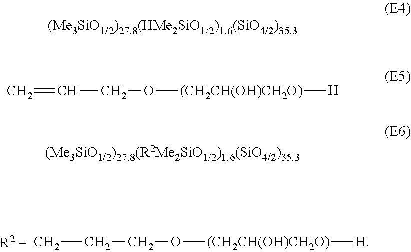

### Examples of Eyebrow Makeup Compositions

The following compositions were prepared:

TABLE 1

| | | | Example 3 |
|---|---|---|---|
| | Example 1 | Example 2 | (outside the |
| Ingredients | (invention) | (invention) | invention) |
| SILICONE RESIN | 18.18 (9% | 18.18 | 0 |
| (3-GLYCEROXYPROPYL) | active | (9% active | |
| DIMETHYLSILOXY | material) | material) | |
| TRIMETHYLSILOXYSILICATE | | | |
| OF FORMULA (21), 50% | | | |
| SOLUTION IN ISODODECANE | | | |
| (X-25-9138A ® from SHIN-ETSU) | | | |
| TRIMETHYLSILOXYSILICATE, | 12.0 | 12.0 | 12.0 |
| 75% SOLUTION IN | (9% active | (9% active | (9% active |
| ISODODECANE | material) | material) | material) |
| (SILSOFT 74 FLUID ®- | | | |
| MOMENTIVE PERFORMANCE | | | |
| MATERIALS) | | | |
| NYLON-611/DIMETHICONE | 12.0 | 0 | 12.0 |
| COPOLYMER | | | |
| (DOWSIL 2-8179 GELLANT ®- | | | |
| DOW CORNING) | | | |
| DISTEARDIMONIUM | 5.0 | 5.0 | 5.0 |
| HECTORITE | | | |
| (BENTONE 38 VCG ® | | | |
| RHEOLOGICAL ADDITIVE- | | | |
| ELEMENTIS) | | | |
| PROPYLENE CARBONATE | 1.65 | 1.65 | 1.65 |
| TITANIUM DIOXIDE (AND) | 2.47 | 2.47 | 2.47 |
| ISOPROPYL TITANIUM | | | |
| TRIISOSTEARATE | | | |
| (BTD-401 ®-KOBO) | | | |
| IRON OXIDES (AND) | 0.59 | 0.59 | 0.59 |
| ISOPROPYL TITANIUM | | | |
| TRIISOSTEARATE | | | |
| (BWRO-12 ®-KOBO) | | | |
| IRON OXIDES (AND) | 0.9 | 0.9 | 0.9 |
| ISOPROPYL TITANIUM | | | |
| TRIISOSTEARATE | | | |
| (BWYO-12 ®-KOBO) | | | |
| IRON OXIDES (AND) | 1.12 | 1.12 | 1.12 |
| ISOPROPYL TITANIUM | | | |
| TRIISOSTEARATE | | | |
| (BWBO-12 ®-KOBO) | | | |
| ISODODECANE | q.s. for 100 | q.s. for 100 | q.s. for 100 |

### Protocol for Preparation of the Compositions

Disteardimonium hectorite was pre-dispersed in isododecane. All the ingredients were added to an Olsa tank, then heated to 70° C. and homogenized for 30 min, and then cooled to room temperature (25° C.).

### Tests for Measuring the Wear Property: Sebum Resistance

### Test Protocol

Each formula 1, 2 and 3 is spread with the manual spreader (100 microns).

The film is left to dry for 24 hours.

Two drops of each solution of artificial sebum were deposited on the film.

The drops are left on the film for 24 hours.

The excess solution is removed by wiping with cotton wool.

The resistance of the film is evaluated (traces of the drop, film dissolved on the edges, holes, etc.)

The film was rubbed at the location where the drops were deposited using a fingerstall.

The resistance of the film is evaluated before and after rubbing according to the following 3 resistance criteria:
A: Good resistance of the film
B: Significant modification of the film
C: Disappearance of the film

The results obtained are indicated in the table below:

TABLE 2

| | Formulation | | | | | |
|---|---|---|---|---|---|---|
| | Example 1 | | Example 2 | | Example 3 | |
| | invention | | invention | | outside the invention | |
| | Sebum resistance | | | | | |
| | Wear | Wear | Wear | Wear | Wear | Wear |
| | property | property | property | property | property | property |
| | before | after | before | after t | before | after |
| | rubbing | rubbing | rubbing | rubbing | rubbing | rubbing |
| Artificial | A | A | A | A | C | C |
| sebum * | | | | | | |

* Artificial sebum:
TRIISOSTEARIN 28.7% by weight
HYDROGENATED POLYISOBUTENE 13.7% by weight
OLEIC ACID 28.0% by weight
OLEYL ERUCATE 22.9% by weight
OCTYLDODECANOL 6.7% by weight

The results of the comparative tests showed that Examples 1 and 2 of the invention comprising the combination of the non-glycerolated silicone resin TRIMETHYLSILOXYSILICATE and the glycerolated silicone resin (3-GLYCEROXYPROPYL) DIMETHYLSILOXY TRIMETHYLSILOXYSILICATE exhibited excellent sebum resistance before and after rubbing, unlike Example 3 outside the invention without glycerolated silicone resin and comprising the non-glycerolated silicone resin TRIMETHYLSILOXYSILICATE combined with the silicone polyamide NYLON-611/DIMETHICONE COPOLYMER.

1. Composition, preferably for caring for and/or making up keratin materials, in particular eyebrows including the eyebrow hairs, the skin for implantation of said hairs and the contours thereof, comprising, in particular in a physiologically acceptable medium:
a) at least one non-glycerolated MQ silicone resin,
b) at least one glycerolated silicone resin;
c) at least one oily phase comprising at least one volatile hydrocarbon oil; and
d) at least one lipophilic thickener.

2.-29. (canceled)

30. Composition according to claim 1, wherein the at least one non-glycerolated MQ silicone resin is a trimethylsiloxysilicate.

31. Composition according to claim 1, wherein the at least one non-glycerolated MQ silicone resin is present in an active material content from 4% to 35% by weight relative to the total weight of the composition.

32. Composition according to claim 1, wherein the glycerolated silicone resin(s) is (are) present in an active material content from 0.1% to 40% by weight relative to the total weight of the composition.

33. Composition according to claim 1, wherein the at least one glycerolated silicone resin contains at least one dimethylsiloxane R(CH₃)₂SiO_{1/2} unit comprising a hydrocarbon radical R comprising a monoglycerol group.

34. Composition according to claim 1, wherein the glycerolated silicone resin(s) is (are) chosen from compounds of formula (I) below: 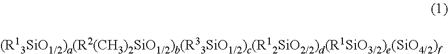
in which
each R¹, which are identical or different, is an alkyl, aryl or aralkyl group with 1 to 30 carbon atoms, or a group substituted by a halogen, a group substituted by an amino or a group substituted by a carboxyl of the latter;
each R² is a monoglycerol or polyglycerol group of general formula (2) below: 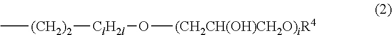
in which
R⁴ is a substituted or unsubstituted monovalent hydrocarbon group or a hydrogen atom, and
the subscripts 1 and i are integers which satisfy the conditions 0≤l≤15 and 0<i≤5,
each R³ is an identical or different group of general formula (3), of general formula (4), of general formula (5) or of general formula (6) below 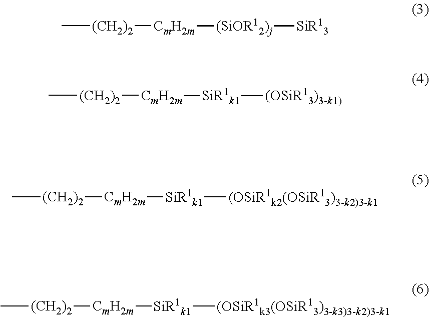
where
each R¹, which are identical or different, is an alkyl, aryl or aralkyl group of 1 to 30 carbon atoms, or a group substituted by a halogen, a group substituted by an amino or a group substituted by a carboxyl of the latter;
the subscripts m, j and k₁ to k₃ are integers which satisfy the conditions 0≤m≤5, 0≤j≤500, 0≤k₁≤2, 0≤k₂≤2 and 0≤k₃≤2; and
the subscripts a, b, c, d, e and f are numbers which satisfy the conditions 0≤a≤400, 0≤b≤200, 0≤c≤400, 0≤d≤320, 0≤e≤320, 0<f≤1000 and 0.5≤(a+b+c)/f≤1.5.

35. Composition according to claim 1, wherein the glycerolated silicone resin(s) is (are) chosen from (3-glyceroxypropyl) dimethylsiloxy trimethylsiloxysilicate compounds corresponding to formula (21) below: 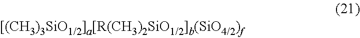
where
R denotes a 3-glyceroxypropyl group of structure
C₃H₆OCH₂—CH(OH)CH₂OH; and
the subscripts a, b and f are integers that satisfy the conditions 0≤a≤400, 0<b≤30, 0<f≤1000 and 0.5≤(a+b)/f≤1.5.

36. Composition according to claim 1, wherein the at least one glycerolated silicone resin and the at least one non-glycerolated MQ silicone resin are present in a weight ratio of the amount of glycerolated silicone resin to the amount of non-glycerolated MQ silicone resin of greater than or equal to 0.8.

37. Composition according to claim 1, wherein the at least one volatile hydrocarbon oil is chosen from C8-C16 isoalkanes.

38. Composition according to claim 1, wherein the volatile hydrocarbon oil(s) is (are), present in contents of less than or equal to 80% by weight relative to the total weight of said composition.

39. Composition according to claim 1, wherein the at least one lipophilic thickener is a lipophilic clay.

40. Composition according to claim 1, wherein the lipophilic thickener(s) is (are) present in concentrations from 0.5% to 10% by weight relative to the total weight of the composition.

41. Composition according to claim 1, further comprising at least one silicone polyamide polymer.

42. Composition according to claim 41, wherein the content of silicone polyamide polymer present in the composition, expressed as active material, is from 5% to 30% by weight relative to the weight of the composition.

43. Composition according to claim 1, further comprising at least one colorant.

44. Composition according to claim 43, wherein the at least one colorant is present in a content of less than or equal to 50.0% by weight relative to the total weight of the composition.

45. Composition according to claim 43, wherein the at least one colorant is chosen from metal oxides.

46. Composition according to claim 43, wherein the at least one colorant is chosen from titanium dioxides coated with isopropyl triisostearyl titanate, black, red or yellow iron oxides coated with isopropyl triisostearyl titanate, and mixtures thereof.

47. Composition according to claim 1, characterized in that it is anhydrous.

48. Assembly, or kit, for packaging and applying a cosmetic composition for coating keratin materials, comprising:
a packaging device comprising the composition as defined in claim 1; and
an applicator for said composition.

49. Method for coating, in particular for curling, keratin materials, in particular the eyebrows and the skin around the eye and eyebrows, comprising the application to said keratin fibers of a composition as defined according claim 1.
